(19) United States
(12) Patent Application Publication
(10) Pub. No.: US 2025/0114437 A1
(43) Pub. Date: Apr. 10, 2025

(54) COMPOSITION FOR USE AS A DIETARY SUPPLEMENT FOR THE PRODUCTION OF SHORT-CHAIN PEPTIDES IN THE DIGESTIVE TRACT WHICH ARE RELEVANT FOR THE APPEARANCE OF THE SKIN

(71) Applicant: QUIRIS HEALTHCARE GMBH & CO. KG, Gütersloh (DE)

(72) Inventor: Peer LANGE, Guetersloh (DE)

(21) Appl. No.: 18/904,608

(22) Filed: Oct. 2, 2024

(30) Foreign Application Priority Data
Oct. 4, 2023 (DE) 102023209700.1

(51) Int. Cl. A61K 38/39; A61K 31/355; A61K 31/375; A61K 31/4188; A61K 33/30; A61K 36/69

(52) U.S. Cl. CPC A61K 38/39; A61K 31/355; A61K 31/375; A61K 31/4188; A61K 33/30; A61K 36/69

(57) ABSTRACT

A composition for use as a dietary supplement for the production of short-chain peptides in the digestive tract which are relevant for the skin appearance is provided, the composition at least includes the components collagen peptides of bovine origin with a molecular mass of 2000 Da on average, which are characterised by a high sequence homology with human collagen type I α-1, type I α-2 and type III α-1 with an overall sequence coverage of greater than 90% for human collagen type I α-1 and α-2 and an aqueous extract of acerola fruit (Malpighia punicifolia), which in addition to the standardised vitamin C content is characterised by the presence of soluble dietary fibre with a high uronic acid content. The disclosed composition is particularly suitable as a dietary supplement for maintaining healthy skin.

### CROSS-REFERENCE TO RELATED APPLICATIONS

This application claims priority from German Patent Application No. 10 2023 209 700.1, filed on Oct. 4, 2023, the entirety of which is hereby fully incorporated by reference herein.

The present invention relates to a composition for use as a food supplement for the production of short-chain peptides in the digestive tract which are relevant for the appearance of the skin, at least comprising bovine collagen peptides having a molecular mass of 2000 Da on average and an extract from the acerola fruit (Malpighia punicifolia).

With increasing age, human skin is subject to visible signs of ageing, which can appear at different times and to varying degrees depending on genetic predisposition and lifestyle factors. These signs of ageing are particularly evident on the face. The use of cosmetic products for decorative purposes has been documented since time immemorial, and in recent decades the skin-caring properties of topical preparations have become increasingly important. However, due to the strong barrier properties of the basal lamina in human skin, the majority of externally applied substances can only have an effect in the outermost skin layer, the epidermis.

For this reason, more and more so-called nutraceuticals have been developed in recent years. These are usually food supplements that contain nutrients relevant to skin health. For example, DE 10 2016 107 508 A1 discloses an oral composition containing collagen hydrolysate and vitamin C for the production of a functional food or a food supplement to promote the maintenance of healthy skin.

In addition to micronutrients such as biotin, zinc, vitamin C, iodine, copper, niacin, riboflavin or vitamin A, which have been recognised by the European Food Safety Authority (EFSA) as having skin-relevant effects in the form of so-called health claims, plant extracts or selected components of the extracellular matrix of animal tissue are also popular ingredients in such products, although the mechanism of action often remains unclear.

A plant extract frequently used in dietary supplements is obtained from the acerola fruit. It is valued for its very high vitamin C content and corresponding extracts for nutritional use are usually (only) standardised to this one ingredient. At most, antioxidant accompanying substances such as polyphenols are sometimes discussed in the literature with regard to their nutritional and physiological effects.

Of the above-mentioned components of the extracellular matrix, the use of collagen peptides is particularly popular. Collagen is the most common structural protein in the human body. The same applies to other vertebrates, so that collagen-containing components are a by-product of meat and fish production that is suitable for human consumption. While the processing of these components into gelatine has a long tradition, the production of collagen peptides has become increasingly important in recent years.

Collagen peptides differ from gelatine both in their galenic properties (e.g. gelling capacity) and in their bioavailability. Which properties significantly influence their effects on skin appearance is currently the subject of scientific research. The original hypothesis that they only provide the body with the amino acids required for protein synthesis (“building block function”) can be regarded as refuted. Instead, there are indications that both the selection of the animal source (bovine, porcine, marine) and the process control in the hydrolysis procedure (e.g. the proteases used) influence the composition and size and thus also the (signalling) effect of the collagen peptides.

While in the past, peptides containing hydroxyproline were considered to be particularly important because they are especially typical of collagen, more recent research suggests that other peptide fragments also have nutritional and physiological significance. The work of the research group led by Zhao Deng et al. has recently shown that peptides containing the sequence PGA (proline-glycine-alanine) are characterised by strong anti-inflammatory activity. In view of the fact that inflammatory processes also contribute to skin ageing, this finding is also of great interest for maintaining skin health.

The disadvantage of the above-mentioned research results is that they are based on synthetically produced peptides or peptide fragments. There is thus a need to investigate the mechanism of action of such preparations in more detail so that the effect of existing preparations can be improved on this basis.

The object of the present invention is to identify a natural-based source of such peptides which can be utilised for nutritional purposes. It is also the object of the present invention to provide these peptides in a composition which has improved properties compared to known compositions for nutritionally influencing the appearance of the skin.

The problem is solved by a composition for use as a food supplement according to claim 1. Further preferred embodiments can be found in the dependent claims.

Surprisingly, by combining collagen peptides with an acerola fruit extract containing certain polysaccharides, it has been possible to influence the peptide fragments formed during human digestion in such a way that preferentially those containing the physiologically relevant sequence PGA are formed.

For the purposes of the present invention, the term “extract” refers to concentrated extracts of fruits or other constituents of plants or of animal constituents of a liquid, viscous, oily or dry nature, which may be adjusted to a specific content of ingredients. Plant-based extracts can be obtained using alcohol or water, for example. For the purposes of the present invention, the term “extract” also refers to purified extracts or extract fractions. For the purposes of the present invention, the term “extract from” is understood to mean that the extract is obtainable or produced from the corresponding plant or plant parts or animal constituents.

According to the invention, bovine collagen peptides with an average molecular weight of 2000 Daltons are used, which are preferably produced by hydrolysis of gelatine from, preferably, bovine skin under the action of various proteases. Characterisation of the raw material by means of LC-MS/MS measurements using a reversed-phase nano LC column shows that the peptides have a chain length of preferably less than 26 peptides.

After analysing the primary structure of the collagen constituents and comparing it with relevant protein databases, a high sequence homology with human collagen type I α-1 of 31%, human collagen type I α-2 of 18% and human collagen type III α-1 of 13% can be determined, which is higher than the sequence coverage of collagen peptides of other origins with 4 to 20%, 7 to 16% and 6 to 11%. The total sequence coverage for human collagen type I α-1 is over 94% and for human collagen type I α-2 over 90%. Due to the content of nutritionally valuable protein and the absence of added preservatives, the collagen constituents according to the invention represent a high-quality food supplement.

The acerola fruit extract used is not only standardised to a fixed content of vitamin C, but is also characterised by a significant content of high-molecular soluble dietary fibre, as can be shown by enzymatic gravimetry. Spectrophotometric methods also reveal a similarly high content of uronic acids. Our own measurements show a dietary fibre content of at least 2.1% (gravimetry) with a high uronic acid content of at least 1.6%. (spectrometry) for the specific acerola fruit extract used here. Uronic acids are monosaccharides of which the primary alcohol functions (CH₂OH group, C6) are oxidised to carboxylic acid functions (COOH group). The molecule therefore has both a carbonyl (aldehyde) function at C1 and a carboxyl function at C6. This leads to the conclusion that the dietary fibres mentioned could be pectin derivatives. It is known that high molecular weight components such as dietary fibres can influence the absorption of other food components in the human intestine.

According to the invention, the aqueous extract from the acerola fruit has the standardised vitamin C content, a content, preferably of 0.2 wt. % to 10.0 wt. %, particularly preferably of 1.0 wt. % to 5.0 wt. % of, preferably high-molecular soluble dietary fibre, and a content, preferably of 0.1 wt. % to 5.0 wt. %, particularly preferably of 0.5 wt. % to 2.5 wt. % of uronic acids (typical/preferred acids should be mentioned at least as examples, so that the invention is disclosed clearly and completely enough to be carried out by a person skilled in the art). The uronic acids are D-glucuronic acid, D-galacturonic acid and D-mannuronic acid. D-galacturonic acid is predominant. The inventors therefore assume that the dietary fibres are pectins. Pectins are polyuronides that substantially consist of α-1,4-glycosidically linked D-galacturonic acid units.

As the nutritional and physiological effects in the skin are determined by the form in which orally ingested ingredients arrive there, the “TinyTIM” model from Triskelion was used to gain a better understanding of the digestive processes. This is a validated in vitro system that mimics the processes in the human digestive tract. Various compartments (gastric, intestinal) are used for this purpose, which are linked by a peristaltic valve. As part of dynamic process control, the conditions in the human gastrointestinal tract are simulated using temperature, pH value, electrolytes, digestive enzymes and mechanics, among other things.

Surprisingly, after digestion of a combination of collagen peptides and acerola extract, various short-chain peptide fragments were detected significantly more frequently than after digestion of gelatine.

Surprisingly, it was shown that many of these peptides contain the amino acid sequence GPA, which is relevant for an anti-inflammatory active component. Among the 10 most frequently occurring peptide fragments, four peptides were found: GPAGPAGPR, GPAGV, GRPGApGPA and VGPA (one-letter code for amino acids; p=hydroxyproline).

Advantageously, the combination of collagen peptides and acerola extract according to the invention, in each case with the described properties, influences the digestion process in the human body in such a way that peptide fragments with the desired amino acid sequence are preferably formed.

The problem formulated at the outset is thus solved by a composition according to claim 1 and its use as a food supplement. Further preferred embodiments can be found in the dependent claims.

In other words, the problem is solved by a composition for use as a food supplement for the production of short-chain peptides in the digestive tract which are relevant for the appearance of the skin, at least comprising the components
a) collagen peptides of bovine origin with an average molecular mass of 2000 Da and
b) aqueous extract of acerola fruit with a standardised vitamin C content, a content of 0.2 wt. % to 10.0 wt. % of high-molecular weight soluble dietary fibres and a content of 0.1 wt. % to 5.0 wt. % of uronic acids.

In a preferred embodiment, the composition further comprises, in each case based on the total weight thereof, at least the components
a) vitamin E in the range of 1×10⁻⁴ wt. % to 4 wt. %, preferably in the range of 4×10⁻⁴ wt. % to 1 wt. %, particularly preferably in the range of 1×10⁻³ wt. % to 1×10⁻² wt. %,
b) biotin in the range of 4×10⁻⁶ wt. % to 0.02 wt. %, preferably in the range of 1×10⁻⁵ wt. % to 1×10⁻³ wt. %, particularly preferably in the range of 1×10⁻⁴ wt. % to 1×10⁻³ wt. % and
c) zinc citrate in the range of 4×10⁻⁴ wt. % to 0.08 wt. %, preferably in the range of 1×10⁻³ wt. % to 1×10⁻¹ wt. %, particularly preferably in the range of 1×10⁻² wt. % to 1×10⁻¹ wt. %, wherein the weight contents of the individual components are selected such that the total weight of the components does not exceed 100 wt. %.

Preferably, the composition contains a) collagen peptides and b) acerola fruit extract in a weight ratio of 8:1 to 2:1, most preferably in a ratio of 3.75:1.

In addition to components a), b) and possibly c) to e), in a preferred embodiment the composition may contain further micronutrients and/or plant extracts as well as galenic excipients (sugars, sweeteners, stabilisers, acidity regulators, preservatives, flavouring agents).

The specification can be readily understood with reference to the following

### Numbered Paragraphs

Numbered Paragraph 1; A composition for use as a food supplement for the production of short-chain peptides in the digestive tract which are relevant for the appearance of the skin, at least containing the components
a) collagen peptides of bovine origin with an average molecular mass of 2000 Da and
b) aqueous extract of acerola fruit with a standardised vitamin C content, a content of 0.2 wt. % to 10.0 wt. % of high-molecular weight soluble dietary fibres and a content of 0.1 wt. % to 5.0 wt. % of uronic acids.

Numbered Paragraph 2: The composition according to Numbered Paragraph 1, characterised in that the collagen peptides have a chain length of less than 26 peptides.

Numbered Paragraph 3: The composition according to Numbered Paragraph 2, characterised in that the collagen peptides have a high sequence homology with the human collagen in the skin of type I α-1, collagen type I α-2 and collagen type III α-1. The total sequence coverage of human collagen type I α-1 is over 94%, for human collagen type I α-2 over 90%.

Numbered Paragraph 4: The composition according to Numbered Paragraph 1, 2 or 3, characterised in that the composition, based on the total weight thereof, further comprises at least the components
c) vitamin E in the range of 1×10⁻⁴ wt. % to 4 wt. %,
d) biotin in the range of 4×10⁻⁶ wt. % to 0.02 wt. % and
e) zinc citrate in the range of 4×10⁻⁴ wt. % to 0.08 wt. %,
wherein the weight contents of the individual components are selected such that the total weight of the components does not exceed 100 wt. %.

Numbered Paragraph 5: The composition according to one of the preceding Numbered Paragraphs, characterised in that a) and b) are contained in a weight ratio of 8:1 to 2:1.

Numbered Paragraph 6: The composition according to any one of the preceding Numbered Paragraphs, characterised in that it contains further micronutrients and/or plant extracts as well as galenic excipients.

Numbered Paragraph 7: The composition according to any one of the preceding Numbered Paragraphs for use as a dietary supplement for maintaining skin health.

Numbered Paragraph 8: The composition according to any one of the preceding Numbered Paragraphs in the administration form of a drinking ampoule.

1. A composition for use as a food supplement for the production of short-chain peptides in the digestive tract which are relevant for the appearance of the skin, at least containing the components
a) collagen peptides of bovine origin with an average molecular mass of 2000 Da and
b) aqueous extract of acerola fruit with a standardised vitamin C content, a content of 0.2 wt. % to 10.0 wt. % of high-molecular weight soluble dietary fibres and a content of 0.1 wt. % to 5.0 wt. % of uronic acids.

2. The composition according to claim 1, wherein the collagen peptides have a chain length of less than 26 peptides.

3. The composition according to claim 2, wherein the collagen peptides have a high sequence homology with the human collagen in the skin of type I α-1, collagen type I α-2 and collagen type III α-1, wherein the total sequence coverage of human collagen type I α-1 is over 94%, and the total sequence coverage for human collagen type I α-2 over 90%.

4. The composition according to claim 1, wherein the composition, based on the total weight thereof, further comprises at least the components
c) vitamin E in the range of 1×10⁻⁴ wt. % to 4 wt. %,
d) biotin in the range of 4×10⁻⁶ wt. % to 0.02 wt. % and
e) zinc citrate in the range of 4×10⁻⁴ wt. % to 0.08 wt. %,
wherein the weight contents of the individual components are selected such that the total weight of the components does not exceed 100 wt. %.

5. The composition according to claim 1, wherein a) and b) are contained in a weight ratio of 8:1 to 2:1.

6. The composition according to claim 1, wherein it contains further micronutrients and/or plant extracts as well as galenic excipients.

7. The composition according to claim 1, for use as a dietary supplement for maintaining skin health.

8. The composition according to claim 1, in the administration form of a drinking ampoule.

9. The composition according to claim 2, wherein the composition, based on the total weight thereof, further comprises at least the components
f) vitamin E in the range of 1×10⁻⁴ wt. % to 4 wt. %,
g) biotin in the range of 4×10⁻⁶ wt. % to 0.02 wt. % and
h) zinc citrate in the range of 4×10⁻⁴ wt. % to 0.08 wt. %,
wherein the weight contents of the individual components are selected such that the total weight of the components does not exceed 100 wt. %.

10. The composition according to claim 3, wherein the composition, based on the total weight thereof, further comprises at least the components
i) vitamin E in the range of 1×10⁻⁴ wt. % to 4 wt. %,
j) biotin in the range of 4×10⁻⁶ wt. % to 0.02 wt. % and
k) zinc citrate in the range of 4×10⁻⁴ wt. % to 0.08 wt. %,
wherein the weight contents of the individual components are selected such that the total weight of the components does not exceed 100 wt. %.
